(19) United States
(12) Patent Application Publication
(10) Pub. No.: US 2024/0238026 A1
(43) Pub. Date: Jul. 18, 2024

(54) ORTHOPEDIC SLIDE HAMMER INSTRUMENT

(71) Applicant: Shukla Medical, St. Petersburg, FL (US)

(72) Inventor: Dustin Saxon, St. Petersburg, FL (US)

(73) Assignee: Shukla Medical, St. Petersburg, FL (US)

(21) Appl. No.: 18/194,275

(22) Filed: Mar. 31, 2023

Related U.S. Application Data
(63) Continuation-in-part of application No. 29/869,961, filed on Jan. 13, 2023.

(51) Int. Cl. A61B 17/92

(52) U.S. Cl. CPC A61B 17/92; A61B 2017/0046; A61B 2017/922

(57) ABSTRACT

A slide hammer including an elongate frame defining a distal end for connection to a tool and a proximal end for connection to a handle. The slide hammer also includes an adapter cap engaged with the proximal end of the elongate frame. The adapter cap also includes a longitudinally extending planar surface. Moreover, the slide hammer also includes a block slidably secured to elongate frame and a handle detachably secured to the adapter cap. The handle includes a recess having a cooperating longitudinally extending planar surface for engaging the longitudinally extending planar surface of the adapter cap for allowing a torsional force applied to the handle to be transferred to the elongate frame.

### BACKGROUND

The present disclosure relates generally to a slide hammer and more particularly to an orthopedic slide hammer instrument having a handle.

Conventional surgical implant removal procedures require the use of tools to assist with removing an implant from the patient, which may be cemented to bone with minimal damage to the surrounding tissue. One common surgical implant removal tool is a slide hammer or slap hammer.

Conventional slide hammers include a mechanism which provides an impact force to dislodge surgical implants from bone. However, conventional slide hammers do not provide other types of force facilitate removal of the implant from bone, particularly torsional force.

Thus, there is a need for a slide hammer which can provide the impact force of a conventional slide hammer along with torsional force to assist with removing surgical implants. Such a need is satisfied by the slide hammer of the present disclosure.

### BRIEF SUMMARY

According to an exemplary embodiment, a slide hammer includes an elongate frame defining a distal end for connection to a tool and a proximal end for connection to a handle. The slide hammer also includes an adapter cap engaged with the proximal end of the elongate frame. The adapter cap also includes a longitudinally extending planar surface. Moreover, the slide hammer also includes a block slidably secured to the elongate frame and a handle detachably secured to the adapter cap. The handle includes a recess having a cooperating longitudinally extending planar surface for engaging the longitudinally extending planar surface of the adapter cap for allowing a torsional force applied to the handle to be transferred to the elongate frame.

In some examples, the elongate frame comprises a rod.

In some examples, the rod includes a threaded proximal end and a threaded distal end.

In some examples, the elongate frame includes a quick connect about its distal end for attaching a tool thereto.

In some examples, the adapter cap includes a pair of opposing longitudinally extending planar surfaces.

In some examples, the adapter cap includes a counterbore for receiving a proximal end of the elongate frame.

In some examples, the adapter cap includes a threaded counterbore for fastening to the proximal end of the elongate frame.

In some examples, the adapter cap is permanently attached to the proximal end of the elongate frame.

In some examples, the adapter cap includes a recessed collar for engaging a lock mechanism.

In some examples, the recess of the handle includes a pair of opposing cooperating longitudinally extending planar surfaces for operatively engaging the pair of opposing longitudinally extending planar surfaces of the adapter cap.

In some examples, the handle further includes a lock mechanism configured to secure the handle to the adapter cap.

In some examples, the lock mechanism includes an engagement collar moveable between a first position and a second position for engaging the adapter cap.

In some examples, the lock mechanism further includes a biasing member configured to bias the engagement collar.

In some examples, the lock mechanism is a quick connection for connecting to the adapter cap.

In some examples, the block is slidable along the elongate frame such that the block is configured to strike the handle.

In some examples, the block includes a recess for receiving the adapter cap therein.

In some examples, the block includes a through hole for receiving the elongate frame therein and a counterbore sized to freely receive the adapter cap therein.

In some examples, the block includes a proximally facing striking surface configured to engage a distally facing striking surface of the handle.

In some examples, the handle is T-shaped handle.

According to another exemplary embodiment, a slide hammer includes a rod defining a distal end connected to a quick connect and a proximal end. The slide hammer also includes an adapter cap permanently connected to the proximal end of the rod. The adapter cap includes a pair of longitudinally extending planar surfaces, and a recess collar. The slide hammer also includes a block slidably secured to the rod. The block includes a through hole for receiving the rod therein and a counterbore sized to freely receive the adapter cap therein. Moreover, the slide hammer includes a T-handle releasably attached to the adapter cap. The T-handle includes a recess having cooperating longitudinally extending planar surfaces for engaging the pair of longitudinally extending planar surface of the adapter cap. Additionally, the slide hammer includes a quick connection for securely attaching to the recess collar of the adapter cap.

### BRIEF DESCRIPTION OF THE SEVERAL VIEWS OF THE DRAWINGS

The foregoing summary, as well as the following detailed description of the exemplary embodiments of the subject disclosure, will be better understood when read in conjunction with the appended drawings. For the purpose of illustrating the invention, there are shown in the drawings exemplary embodiments. It should be understood, however, that the present disclosure is not limited to the precise arrangements and instrumentalities shown.

In the drawings:

FIG. 1 is a perspective view of a slide hammer according to an exemplary embodiment of the subject disclosure;

FIG. 2 is an exploded side view of the slide hammer of FIG. 1;

FIG. 3 is a partial cross-sectional view of the slide hammer of FIG. 1;

FIG. 4 is a perspective view of the slide hammer of FIG. 1 having a handle removed for purposes of illustration;

FIG. 5 is a longitudinal cross-sectional view of the slide hammer in FIG. 4;

FIG. 6 is a front elevation view of an adapter cap of the slide hammer of FIG. 1;

FIG. 7 is a side elevation view of the adapter cap of FIG. 6;

FIG. 8 is a bottom perspective view of the adapter cap of FIG. 6;

FIG. 9 is a top plan view of the adapter cap of FIG. 6;

FIG. 10 is a bottom plan view of the adapter cap of FIG. 6;

FIG. 11 is a cross-sectional view of the slide hammer of FIG. 1 having an engagement structure in a first position;

FIG. 12 is a cross-sectional view of the slide hammer of FIG. 1 having the engagement structure in a second position;

FIG. 13 is a bottom perspective view of the handle of the slide hammer of FIG. 1; and

FIG. 14 is a perspective view of a lock mechanism of the slide hammer of FIG. 1; and

FIG. 15 is a bottom perspective view of a quick connect mechanism of the slide hammer of FIG. 1.

### DETAILED DESCRIPTION

Reference will now be made in detail to the various exemplary embodiments of the subject disclosure illustrated in the accompanying drawings. Wherever possible, the same or like reference numbers will be used throughout the drawings to refer to the same or like features. It should be noted that the drawings are in simplified form and are not drawn to precise scale. Certain terminology is used in the following description for convenience only and is not limiting. Directional terms such as top, bottom, left, right, above, below and diagonal, are used with respect to the accompanying drawings. The term “distal” shall mean away from the center of a body. The term “proximal” shall mean closer towards the center of a body and/or away from the “distal” end. The words “inwardly” and “outwardly” refer to directions toward and away from, respectively, the geometric center of the identified element and designated parts thereof. Such directional terms used in conjunction with the following description of the drawings should not be construed to limit the scope of the subject disclosure in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, or ±0.1% from the specified value, as such variations are appropriate.

“Substantially” as used herein shall mean considerable in extent, largely but not wholly that which is specified, or an appropriate variation therefrom as is acceptable within the field of art. “Exemplary” as used herein shall mean serving as an example.

Throughout this disclosure, various aspects of the subject disclosure can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages, and characteristics of the exemplary embodiments of the subject disclosure may be combined in any suitable manner in one or more embodiments. One skilled in the relevant art will recognize, in light of the description herein, that the present disclosure can be practiced without one or more of the specific features or advantages of a particular exemplary embodiment. In other instances, additional features and advantages may be recognized in certain embodiments that may not be present in all exemplary embodiments of the subject disclosure.

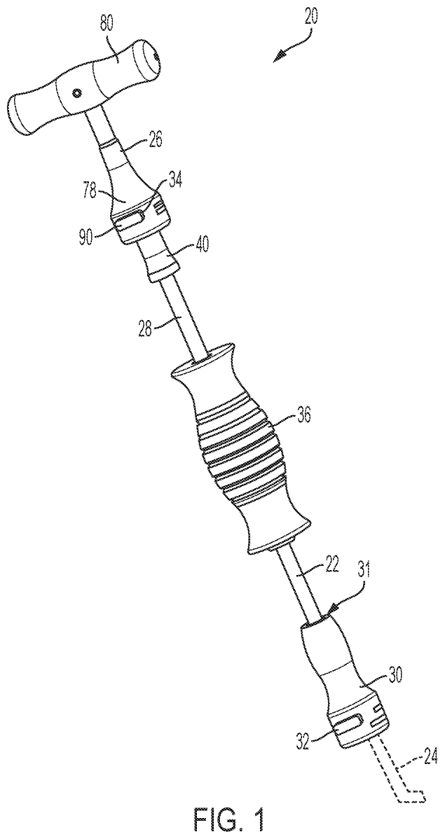

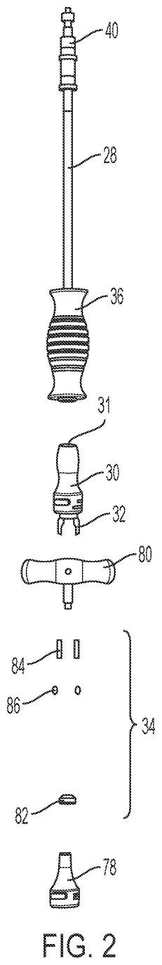

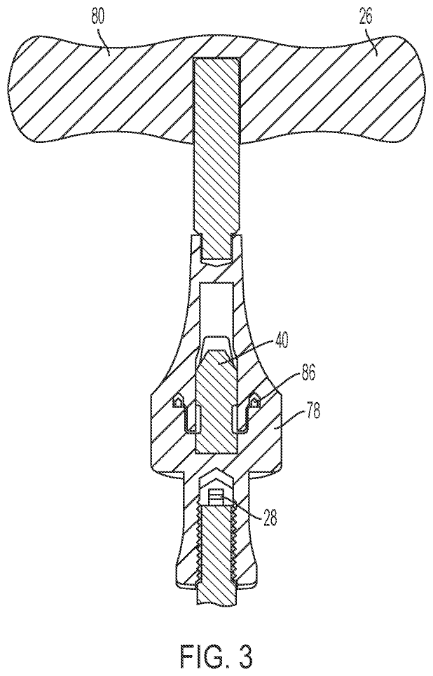

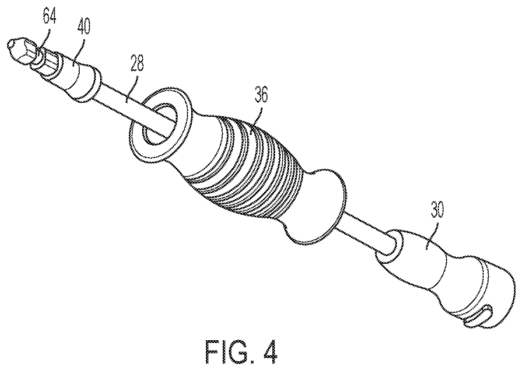

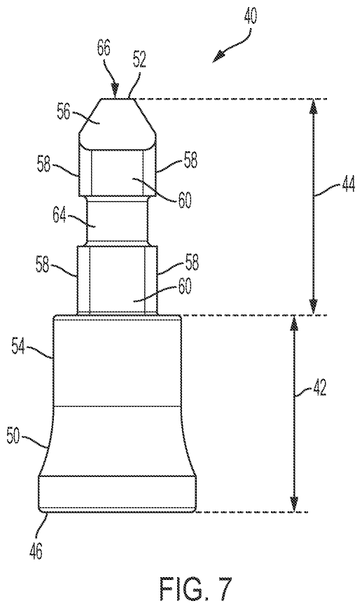

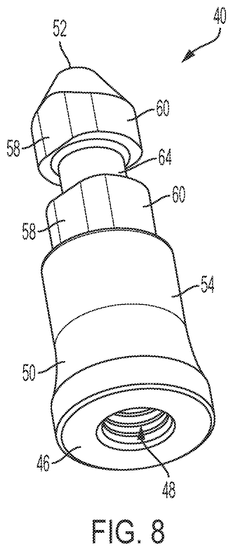

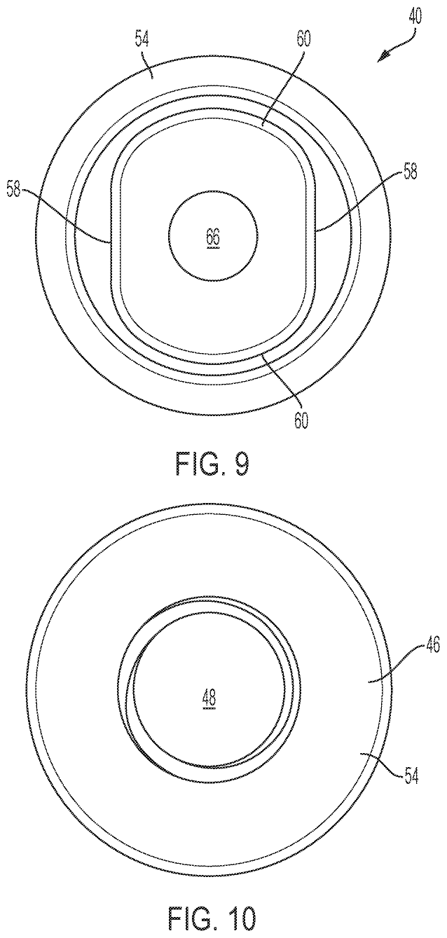

In accordance with an exemplary embodiment, the subject disclosure provides a slide hammer 20 as configured in FIGS. 1-15. The slide hammer 20 includes an elongate frame 22, an adapter cap 40, a block 36 and a handle 26.

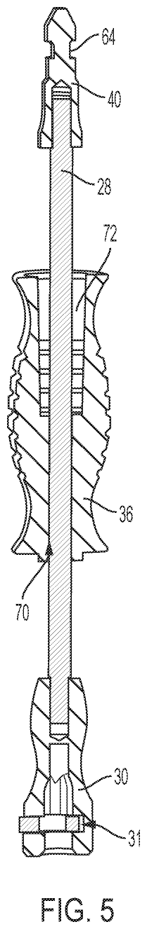

The elongate frame 22 is best shown in FIGS. 1, 4 and 5, and includes a distal end for connection to a tool 24 and a proximal end for connection to the handle 26. The elongate frame 22 includes a rod 28 extending between the distal end and the proximal end. Moreover, the rod 28 includes a threaded proximal end and a threaded distal end (FIG. 5) to facilitate coupling to the handle 26 and the tool 24. In some examples, the elongate rod 28 is generally cylindrical and configured to have sufficient length to allow the block 36 to be slidably engaged thereon.

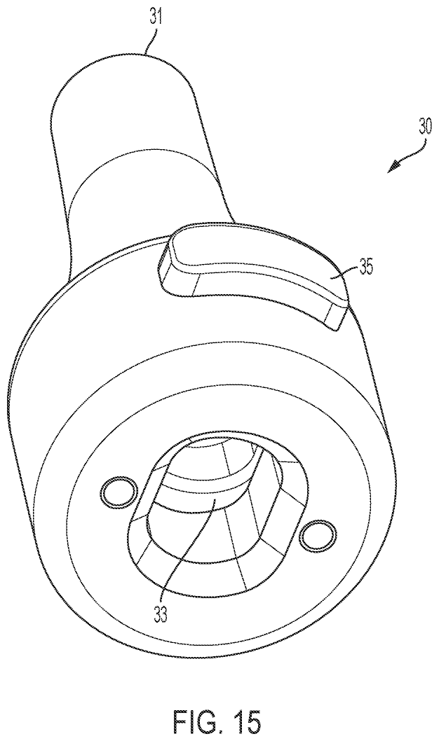

The distal end of the elongate frame 22 includes a quick connect mechanism 30 for attaching the tool 24 thereto. Referring to FIG. 15, the quick connect mechanism 30 includes a counterbore 31 configured to be coupled to the threaded distal end of the elongate frame. Once connected via the threads, the treaded connection can be permanently secured e.g., via a weld, adhesive or other means.

The quick connect mechanism 30 also includes a bore 33 at its distal end which is configured to receive the tool 24. Within the bore 33 is a tool quick connect 35 securing the tool thereto.

The tool 24 may be any tool 24 including but not limited to tools configured for impacting, cutting, incising, dissecting, grasping, holding or the like. Such tools are correspondingly configured to include a cooperating tool quick connect for releasably connecting to the tool quick connect 35.

Referring back to FIG. 1, the proximal end of the elongate frame 22 is configured for connection to the handle 26. In the example shown, the handle 26 is a T-handle 26 having an overall shape or profile of a T i.e., T-shaped, however, the handle could alternatively be configured to have shapes other than T-shaped e.g., ball-shaped, circular-shaped, and the like as desired. Additional details regarding the handle are further discussed below.

To aid in coupling of the elongate frame 22 to the handle 26 such that a multitude of forces can be transmitted, the slide hammer 20 also includes the adapter cap 40 configured to be engaged with the proximal end of the elongate frame 22. The adapter is configured as best shown in FIGS. 1, and 4-12. In the exemplary embodiment shown, the adapter cap 40 includes a first portion 42 which is configured to be engaged with the proximal end of the elongate frame and a second portion 44 configured to be engaged with the handle 26.

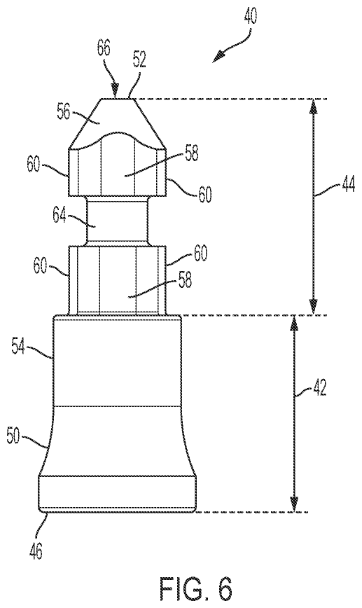

As best shown in FIGS. 6-10, the first portion 42 includes a first end 46 and includes a counterbore 48 for receiving the proximal end of the elongate frame 22. In some examples, the counterbore 48 is threaded for fastening to the proximal end of the elongate frame 22. In the example shown, the counterbore 48 of the first portion 42 is a threaded counterbore 48 configured to receive the proximal end of the elongate frame 22. However, the first portion can alternatively be configured with other fastening mechanisms suitable for connecting to the elongate frame, e.g., a tapered lock, a set screw, a quick connection, a cam lock, and other coupling mechanisms known in the art and suitable for the intended purpose. Additionally, in some examples, the adapter cap 40 and the proximal end of the elongate frame 22 are permanently attached. The permanent attachment may be through welding, adhesive, or other known methods and may be used in conjunction with or as an alternative to the threaded counterbore 48.

Moreover, as shown in the example in FIGS. 6-10, the first portion 42 is generally cylindrical in shape and also includes a tapered portion 50 (e.g., a frustum shaped portion) such that a diameter of the cylinder at the first end 46 of the adapter cap 40 has a larger diameter than the cylinder i.e., base portion 54, adjacent the second portion 44 of the adapter cap 40. In the example shown, an outer surface of the first portion 42 is generally smooth and free from protrusions, apertures, or indentations, however, it is contemplated that one or more protrusions, apertures, indentations or other markings may be present on the outer surface of the first portion 42 of the adapter cap 40 without departing from the spirit of the invention.

The second portion 44 of the adapter cap 40 includes a second end 52 and is configured to engage or releasably engage the handle 26. The second portion 44 of the adapter cap 40 extends from the base portion 54 of the first portion 42 of the adapter cap 40. The second end 52 includes a tapered tip portion 56 and a recessed collar 64. The adapter cap 40 also includes at least one longitudinally extending planar surface 58. In the example shown, the adapter cap 40 includes a pair of opposing longitudinally extending planar surfaces 58. Each longitudinally extending planar surface faces laterally and define a plane substantially parallel to a longitudinal axis of the adapter cap. The pair of opposing longitudinally extending planar surfaces 58 are configured to allow a torsional force applied to the handle 26 to be transferred thereto and further on to the elongate frame 22. Longitudinally extending curved surfaces 60 extends between the pair of opposing longitudinally extending planar surfaces 58. As shown in FIGS. 6-10, the pair of opposing longitudinally extending planar surfaces 58 and the pair of opposing longitudinally extending curved surfaces 60 extend from the base portion 54 to the tapered tip portion 56. The length of these surfaces is configured to correspond or cooperate with a corresponding handle attachment quick connect.

Referring still to the example shown in FIGS. 6-10, the recessed collar 64 is configured for engaging lock mechanism 34 (e.g., a quick connect) of a handle to be attached the adapter cap. The recessed collar 64 is disposed about a mid-portion of the second portion 44 of the adapter cap 40. In the example shown, the recessed collar 64 is an annular recess such that the recessed collar 64 has a smaller overall diameter than the remainder of the second portion 44 of the adapter cap 40. Additionally, in the example shown, the recessed collar is a generally smooth curved surface, however, it has been contemplated that the recessed collar may include protrusions, apertures, indentations, or the like without departing from the spirit of the subject disclosure.

The block 36 is configured as best shown in FIGS. 1 and 5 and is slidably secured to the elongate frame 22. The block 36 is slidable along the elongate frame 22 such that the block 36 can strike the handle 26 attached to the elongate frame to provide impact forces for removing a surgical implant from bone. The block 36 may be any size, shape, or weight, as desired to provide the requisite or predetermined force or momentum handle for imparting such forces or momentum to the surgical implant to be removed. The block 36 has a main body having a through hole 70 for receiving the elongate frame 22 therein. The through hole 70 is sufficiently sized and shaped to receive the elongate frame and to allow the block 36 to slide along the elongate frame between its distal end and proximal end. The block 36 includes a proximally facing striking surface 37 configured to engage a distally facing striking surface of the handle 26 and a distally facing striking surface 39 for engaging the quick connection mechanism 30 about the distal end of the elongate frame. To allow the striking surface of the block 36 to engage the striking surface of the handle 26, the block 36 defines a counterbore 72 for receiving the adapter cap 40 therein. The counterbore 72 may be sized and shaped to freely receive the adapter cap 40 therein which allows the striking surface of the block 36 to engage the striking surface of the handle 26 without engaging or imparting impact forces to the adapter cap.

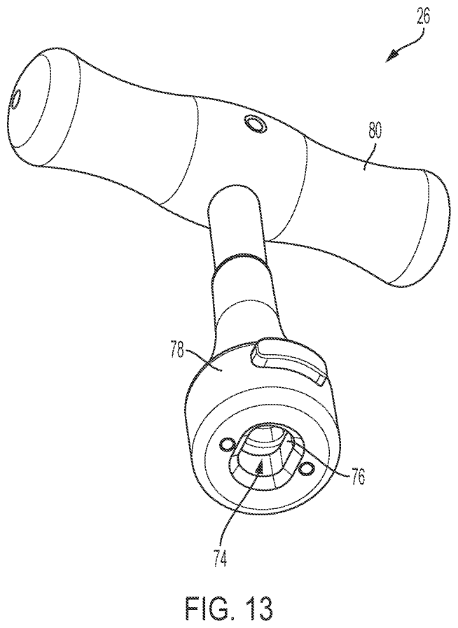

Referring to FIGS. 1 and 13, the handle 26 is configured to be detachably secured to the adapter cap 40. In one example the handle 26 includes a recess 74 for receiving the adapter cap 40. The recess 74 includes at least one longitudinally extending planar surface 76 for engaging the longitudinally extending planar surface 58 of the adapter cap 40 for allowing a torsional force applied to the handle 26 to be transferred to the elongate frame 22. More particularly, the longitudinally extending planar surface 76 facingly engages the longitudinally extending planar surface 58 of the adapter cap. In the example shown, the recess 74 includes two opposing longitudinally extending planar surfaces 76 which operably engage the cooperating longitudinally extending planar surfaces 58 of the adapter cap 40 for transmitting a torsional force applied to the handle 26 to the elongate frame 22. In other words, the engagement or facing alignment of the longitudinally extending planar surfaces 58, 72 of the recess 74 of the handle 26 and of the adapter cap 40 prevents rotation between the elongate frame 22 and the handle 26 such that torsional force can be transferred from the handle 26 to the tool 24.

The handle 26 includes a base 78 having the lock mechanism 34 and configured to receive the adapter cap 40, and a grip portion 80 configured to be engaged by a user's hand. The base 78 is generally cylindrical having a tapered portion such that the base 78 tapers from an end configured to be coupled to the adapter cap 40 to an end coupled to the grip portion 80. The base 78 generally has a smooth surface, although the portion of the base 78 which houses the lock mechanism 34 may define a plurality of apertures to accommodate the same.

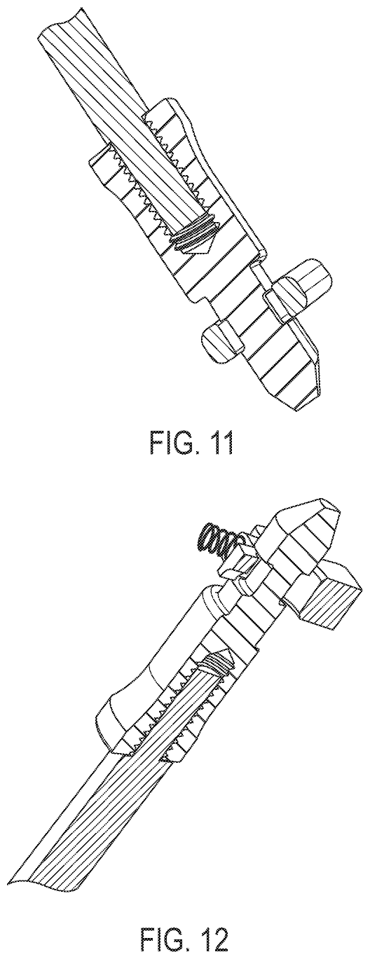

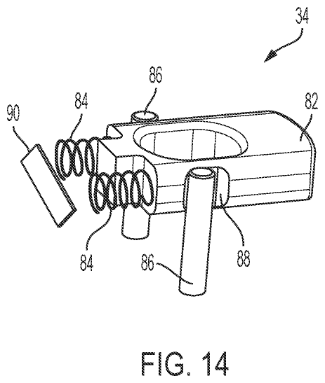

As best shown in FIGS. 13 and 14, the lock mechanism 34 of the handle is configured to secure the handle 26 to the adapter cap 40. In some examples, the lock mechanism 34 is a quick connection lock mechanism 34 for connecting to the adapter cap 40. In some examples the lock mechanism 34 is a guillotine lock. In the example shown in FIGS. 13 and 14, the lock mechanism 34 includes an engagement structure configured to engage the adapter cap 40 to secure the handle 26 and the adapter cap 40 to an engagement collar 82. The engagement structure is moveable between a first position or engaged position (see FIG. 11) and a second position or disengaged position (see FIG. 12) for engaging the recessed collar of the adapter cap 40. In the example shown, the engagement structure is an engagement collar 82 having a generally rectangular shape and defining a generally oval aperture therethrough. In the first position, the aperture of the engagement collar 82 is disposed off-center within the recess 74 of the handle 26 such that the tip surface 66 of the adapter cap 40 would be prevented from fully entering the recess 74 of the handle 26. In the second position, the aperture of the engagement collar 82 is moved toward the center of the recess 74 such that the tip surface 66 of the adapter cap 40 may be disposed therethrough.

The lock mechanism 34 further includes a biasing member 84 configured to bias the engagement collar 82 in one of the first position or the second position. In the example shown, the biasing member 84 is configured to bias the engagement collar 82 in the first position. Additionally, in the example shown, the biasing member 84 includes two biasing members 84 disposed on either side of the oval aperture, however, various other quantities and configurations of biasing members 84 have been contemplated. The biasing member(s) 84 may be any type of biasing member 84 including but not limited to a compression spring, a torsion spring, an extension spring or the like.

In some examples, the lock mechanism 34 further includes a dowel pin 86 configured to prevent undesired movement of the engagement collar 82. More specifically, in the example shown, the engagement collar 82 defines a pair of opposing indents 88 disposed on either side of the oval aperture. Each of the indents 88 are configured to engage a dowel pin 86 to facilitate movement between the first position and the second position. In the example shown, the dowel pin 86 is a generally cylindrical dowel pin 86 which extends on either side of the recess 74 of the handle 26. That is, each dowel pin has a central longitudinal axis that extends transverse to a major plane of the engagement collar 82. The dowel pins 86 secure the engagement collar 82 in place and prevent undesired lateral and rotational movement of the engagement collar 82.

The lock mechanism 34 also includes a push button 90 disposed on the outer surface of the handle 26 and configured to move the engagement collar 82 between the first position and the second position. More specifically, when the push button 90 is pressed by a user, the force of the user engaging the biasing member 84 moves the engagement collar 82 against the bias and into the second position. During movement by the engagement collar 82 the indents 88 of the engagement collar 82 slidably engage the stationary dowel pins. When the push button 90 is released, the biasing member 84 once again biases the engagement collar 82 into the first position.

In operation, the slide hammer 20 includes the elongate frame 22 having the rod 28 with the quick connection configured to be coupled to a tool rotatably coupled to the distal end of the rod 28 and an adapter cap 40 coupled to the proximal end of the rod 28. When use of the slide hammer 20 is needed, a user will couple the handle to the adapter cap 40 by engaging the adapter cap 40 with the recess 74 of the handle 26 while engaging the push button 90. To do so, the user initially aligns the planar surfaces 76 of the handle with the planar surfaces 58 of the adapter cap so as to allow the second portion of the adapter cap to be inserted into the recess 74. In doing so, the user engages the push button 90 to move the engagement collar 82 against the bias and into the second position. During movement by the engagement collar 82 the indents 88 of the engagement collar 82 slidably engage the stationary dowel pins. When the engagement collar 82 is in the second position the adapter cap 40 is able to be inserted through the engagement collar 82. When the push button 90 is released, the biasing member 84 once again biases the engagement collar 82 into the first position and the engagement collar 82 engages the recessed collar 64 of the adapter cap 40 to secure the adapter cap 40 in the longitudinal direction. Additionally, when the adapter cap 40 and the handle 26 are coupled, the engagement of the longitudinally extending surfaces of the recess 74 of the handle 26 and of the adapter cap 40 prevent rotation between the elongate frame 22 relative to the handle 26 such that torsional forces applied to the handle can be transferred from the handle 26 to the tool 24.

Conventional slide hammers include a mechanism which provides an impact force to provide linear pull-out forces for removing surgical implants from bone. However, conventional slide hammers do not provide other types of force that can significantly improve removal of an implant from bone, particularly torsional force. Thus, the slide hammer 20 as described herein has an advantage over conventional slide hammers as the slide hammer 20 as described herein provides the typical impact force of a slide hammer along with an additional torsional force to assist with removing the surgical implant from bone and which eliminates the need for an additional torsional force tool.

It will be appreciated by those skilled in the art that changes could be made to the various exemplary embodiments described above without departing from the broad inventive concept thereof. It is to be understood, therefore, that the subject disclosure is not limited to the particular aspects disclosed, but it is intended to cover modifications within the spirit and scope of the subject application as defined by the appended claims and disclosed above.

I/We claim:

1. A slide hammer comprising:
an elongate frame defining a distal end for connection to a tool and a proximal end for connection to a handle;
an adapter cap engaged with the proximal end of the elongate frame, the adapter cap including a longitudinally extending planar surface;
a block slidably secured to the elongate frame; and
a handle detachably secured to the adapter cap, the handle including a recess having a cooperating longitudinally extending planar surface for engaging the longitudinally extending planar surface of the adapter cap for allowing a torsional force applied to the handle to be transferred to the elongate frame.

2. The slide hammer of claim 1, wherein the elongate frame comprises a rod.

3. The slide hammer of claim 2, wherein the rod includes a threaded proximal end and a threaded distal end.

4. The slide hammer of claim 1, wherein the elongate frame includes a quick connect about its distal end for attaching a tool thereto.

5. The slide hammer of claim 1, wherein the adapter cap includes a pair of opposing longitudinally extending planar surfaces.

6. The slide hammer of claim 1, wherein the adapter cap includes a counterbore for receiving a proximal end of the elongate frame.

7. The slide hammer of claim 1, wherein the adapter cap includes a threaded counterbore for fastening to the proximal end of the elongate frame.

8. The slide hammer of claim 1, wherein the adapter cap is permanently attached to the proximal end of the elongate frame.

9. The slide hammer of claim 1, wherein the adapter cap includes a recessed collar for engaging a lock mechanism.

10. The slide hammer of claim 5, wherein the recess of the handle includes a pair of opposing cooperating longitudinally extending planar surfaces for operatively engaging the pair of opposing longitudinally extending planar surfaces of the adapter cap.

11. The slide hammer of claim 1, wherein the handle further includes a lock mechanism configured to secure the handle to the adapter cap.

12. The slide hammer of claim 11, wherein the lock mechanism includes an engagement collar moveable between a first position and a second position for engaging the adapter cap.

13. The slide hammer of claim 12, wherein the lock mechanism further includes a biasing member configured to bias the engagement collar.

14. The slide hammer of claim 11, wherein the lock mechanism is a quick connection for connecting to the adapter cap.

15. The slide hammer of claim 1, wherein the block is slidable along the elongate frame such that the block is configured to strike the handle.

16. The slide hammer of claim 1, wherein the block includes a recess for receiving the adapter cap therein.

17. The slide hammer of claim 1, wherein the block includes a through hole for receiving the elongate frame therein and a counterbore sized to freely receive the adapter cap therein.

18. The slide hammer of claim 1, wherein the block includes a proximally facing striking surface configured to engage a distally facing striking surface of the handle.

19. The slide hammer of claim 1, wherein the handle is T-shaped handle.

20. A slide hammer comprising:
a rod defining a distal end connected to a quick connect and a proximal end;
an adapter cap permanently connected to the proximal end of the rod, the adapter cap including:
a pair of longitudinally extending planar surfaces, and
a recess collar;
a block slidably secured to the rod, the block including:
a through hole for receiving the rod therein and a counterbore sized to freely receive the adapter cap therein; and
a T-handle releasably attached to the adapter cap, the T-handle including:
a recess having cooperating longitudinally extending planar surfaces for engaging the pair of longitudinally extending planar surface of the adapter cap, and
a quick connection for securely attaching to the recess collar of the adapter cap.
